(19) United States
(12) Patent Application Publication
(10) Pub. No.: US 2026/0024669 A1
(43) Pub. Date: Jan. 22, 2026

(54) IDENTIFYING CORE PATIENTS IN PATIENT CLUSTERS USING MACHINE LEARNING

(71) Applicant: NEUMORA THERAPEUTICS, INC., Watertown, MA (US)

(72) Inventor: Tathagata Banerjee, Waltham, MA (US)

(21) Appl. No.: 19/262,841

(22) Filed: Jul. 8, 2025

Related U.S. Application Data
(60) Provisional application No. 63/672,619, filed on Jul. 17, 2024.

(51) Int. Cl. G16H 50/70; G06N 3/0455; G06N 20/20

(52) U.S. Cl. CPC G16H 50/70; G06N 3/0455; G06N 20/20

(57) ABSTRACT

Methods, systems, and apparatus, including computer programs encoded on a computer storage medium, for processing biomedical data of a plurality of patients. The system selects, for each patient cluster in a set of patient clusters, a proper subset of a plurality of patients included in the patient cluster as core patients based on centrality scores of the patients in the patient cluster. The system outputs data identifying: (i) the set of patient clusters, and (ii) the core patients for each patient cluster.

### CROSS-REFERENCE TO RELATED APPLICATION

This application claims priority to U.S. Provisional Application No. 63/672,619, filed on Jul. 17, 2024. The disclosure of the prior application is considered part of and is incorporated by reference in the disclosure of this application.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a machine learning system implemented as computer programs on one or more computers in one or more locations for processing multi-modal data characterizing patients.

Throughout this specification, a data “modality” refers to a type of data, e.g., that is generated using a specified sensor or medical diagnostic technique, and “multi-modal” data refers to a collection of data from multiple different modalities. An “embedding” refers to an ordered collection of numerical values, e.g., a vector, matrix, or other tensor of numerical values.

According to one aspect, there is provided a method performed by one or more computers, the method comprising: selecting, for each patient cluster in a set of patient clusters, a proper subset of a plurality of patients included in the patient cluster as core patients for the patient cluster, comprising: generating, for each patient included in the patient cluster, a centrality score characterizing how closely the patient is associated with the patient cluster based on a biomedical data embedding associated with the patient; and selecting the proper subset of the plurality of patients included in the patient cluster as core patients for the patient cluster based on the centrality scores; and outputting data identifying: (i) the set of patient clusters, and (ii) the core patients for each patient cluster.

In some implementations, the set of patient clusters are generated by operations comprising: receiving, for each patient in a population of patients, a set of biomedical data characterizing the patient; processing, for each patient in the population of patients, the set of biomedical data characterizing the patient using an encoder machine learning model to generate a biomedical data embedding of the set of biomedical data in a latent space; and clustering the patients in the population of patients, based on the respective biomedical data embedding associated with each patient, to identify the set of patient clusters.

In some implementations, the method further comprises: determining, for each patient, a measure of stability of an assignment of the patient to the patient cluster that includes the patient over a plurality of instances of clustering; wherein for each patient in each patient cluster, the centrality score for the patient is based at least in part on the stability of the assignment of the patient to the patient cluster that includes the patient.

In some implementations, determining, for each patient, the measure of stability of the assignment of the patient to the patient cluster that includes the patient over the plurality of instances of the clustering comprises: performing the plurality of instances of the clustering, wherein each instance of the clustering generates a respective set of patient clusters; and determining, for each patient, the measure of stability based on a measure of overlap between the patient clusters that include the patient over the plurality of instances of the clustering.

In some implementations, the method further comprises: training a discriminative machine learning model to process data characterizing a patient to generate a discriminative output that classifies the patient as being included in a respective one of the patient clusters from the set of patient clusters; wherein for each patient in each patient cluster, generating the centrality score for the patient comprises: determining a confidence measure that characterizes a confidence of the trained discriminative machine learning model in classifying the patient as being included in the patient cluster; and determining the centrality score for the patient based on the confidence measure.

In some implementations, the method further comprises: determining, for each patient cluster, parameters of a distribution function that characterizes a distribution of biomedical data embeddings of patients included in the patient cluster; and determining, for each patient in each patent cluster, the centrality score for the patient based at least in part on a likelihood of the biomedical data embedding of the patient under the distribution function for the patient cluster.

In some implementations, the method further comprises: determining, for each patient cluster, a centroid of biomedical data embeddings of patients included in the patient cluster; and determining, for each patient in each patient cluster, the centrality score for the patient based at least in part on a distance between: (i) the biomedical data embedding of the patient, and (ii) the centroid of the patient cluster that includes the patient.

In some implementations, the method further comprises: generating a set of training examples based on only core patients in the population of patients, wherein: each training example corresponds to a core patient from the population of patients; each training example comprises: (i) a training input that includes the set of biomedical data characterizing the core patient, and (ii) a target output that includes a label that identifies the patient cluster that includes the core patient; and training a classification machine learning model on the set of training examples.

In some implementations, the method further comprises: receiving a set of biomedical data characterizing a new patient; processing the set of biomedical data characterizing the new patient using the classification machine learning model to classify the new patient as being included in a patient cluster from the set of patient clusters.

In some implementations, the method further comprises: generating a recommendation for clinical treatment of the new patient based at least in part on the classification of the new patient generated using the classification machine learning model.

In some implementations, the method further comprises: administering a drug to the new patient based at least in part on the classification of the new patient generated using the classification machine learning model.

In some implementations, the classification machine learning model is trained subject to a constraint that classifications generated by the classification machine learning model depend on at most a predefined, maximum number of biomedical features.

In some implementations, the maximum number of biomedical features is two, or three, or four, or five.

In some implementations, the classification machine learning model is a decision tree, and the constraint defines a maximum depth of the decision tree.

In some implementations, the method further comprises: determining, for each patient cluster, a set of statistics that characterize the patient cluster based on only the core patients of the patient cluster.

In some implementations, the encoder machine learning model comprises an encoder neural network.

In some implementations, the encoder neural network has been trained by operations comprising, for each of a plurality of training patients: processing a set of biomedical data characterizing the training patient using the encoder neural network to generate an embedding in a latent space; processing the embedding using a decoder neural network to generate a reconstruction of the set of biomedical data characterizing the training patient; and training the encoder neural network and the decoder neural network to optimize an objective function that measures an error in the reconstruction of the set of biomedical data characterizing the training patient.

In some implementations, for each patient, the set of biomedical data characterizing the patient comprises respective feature dimensions representing each of a plurality of modalities.

In some implementations, the plurality of modalities include one or more of: (i) a functional magnetic resonance imaging (fMRI) modality, wherein the feature dimensions representing the fMRI modality are derived from a series of fMRI images that each correspond to a respective time point in a sequence of time points and characterize blood flow in a brain of a subject at the time point; or (ii) a genomics modality, wherein the feature dimensions representing the genomics modality are derived from data defining a sequence of nucleotides from a genome of a subject; or (iii) an electroencephalography (EEG) modality, wherein the feature dimensions representing the EEG modality are derived from a plurality of voltage waveforms that are each measured by a respective electrode placed in proximity to a brain of a subject; or (iv) an audio modality, wherein the feature dimensions representing the audio modality are derived from audio data that represents a sequence of words spoken by a subject; or (v) a proteomic modality, wherein the feature dimensions representing the proteomic modality are derived from proteomic data that represents expression levels of proteins in a subject.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect, there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

The machine learning system described in this specification can be used to process multi-modal data characterizing a population of patients to partition the patients into a set of patent clusters, and identify a subset of core patients for each patient cluster. Each respective patient cluster can include a set of patients that belong to a corresponding patient category. Each patient category can be understood as to represent a “type” of patient, e.g., such that patients included in the same patient category are more likely to share similar characteristics. The core patients for the respective patient cluster can include patients that are most representative of the corresponding patient category.

The patient categories identified by the machine learning system can be used as a basis for making inferences (predictions) about patients and for making clinical decisions related to patient care. For example, the patient categories can be used to identify types of patients that are more likely to respond well to certain medical treatments, as will be described in more detail below.

The core patients identified by the machine learning system can be used in downstream analysis to focus on the more representative patients in each patient category, potentially leading to more efficient and accurate analysis. For example, the data of the identified core patients can be used as training examples for training a classification machine learning model for classifying a new patient (e.g., assigning a patient category to the new patient) based on their biomedical data. By limiting the training examples to the core patients (rather than using the entire patient population as training data), the described system reduces the number of training examples required, leading to faster training times and potentially lower computational demands. Furthermore, core patients, by capturing the essential characteristics of their respective clusters, provide a more generalized representation of the overall patient population within that cluster. Training with this data allows the model to learn patterns that are more likely to generalize well to unseen patients belonging to the same cluster, leading to more accurate classification. That is, by using training data comprising data from smaller, more representative groups, the system can achieve more efficient training and potentially more accurate classification results.

In some cases, the classification model can be an interpretable machine learning model (e.g., a decision tree) that focuses on a limited number of features. The interpretable model can provide the reasoning behind the model's predictions and can provide clinically relevant insights in the model's predictions. By highlighting the key features that distinguish patient clusters, the classification model can guide further research and potentially lead to the development of more targeted treatment approaches that address the underlying factors identified through classification. Focusing on a smaller set of features significantly reduces the computational complexity of training the model. This translates to faster training times and lower computational costs, making the analysis process more efficient and potentially applicable in resource-constrained settings.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example machine learning system.

FIG. 2 shows an example patent clustering system.

FIG. 3 shows an example encoder training system.

FIG. 4 is a flow diagram of an example process for determining core patients for each of a set of patient clusters.

FIG. 5 is a flow diagram of an example process for determining patent clusters.

FIG. 6A is a flow diagram of an example process for determining patient centrality scores.

FIG. 6B is a flow diagram of another example process for determining patient centrality scores.

FIG. 6C is a flow diagram of another example process for determining patient centrality scores.

FIG. 6D is a flow diagram of another example process for determining patient centrality scores.

FIG. 7 is a flow diagram of an example process of training a patient classification machine learning model.

FIG. 8 is a flow diagram of an example process for training an encoder machine learning model.

FIG. 9 is a flow diagram of an example process for classifying a patient.

Like reference numbers and designations in the various drawings indicate like elements.

### DETAILED DESCRIPTION

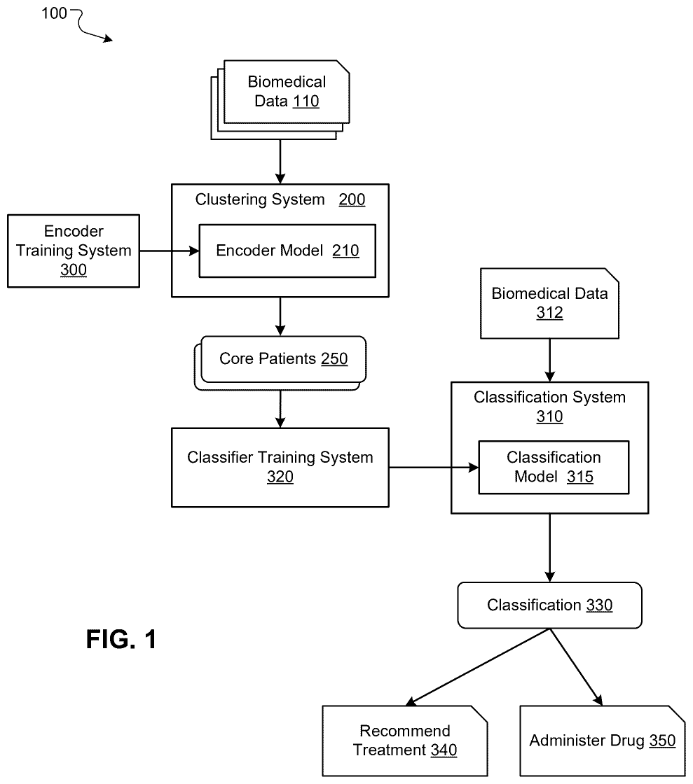

FIG. 1 shows an example machine learning system 100. The machine learning system 100 is an example of a system implemented as computer programs on one or more computers in one or more locations in which the systems, components, and techniques described below are implemented.

The machine learning system 100 processes biomedical data 110 characterizing patients. In some implementations, the biomedical data 110 includes, for each patient, multi-modal data that includes a respective feature representation for each modality in a set of multiple modalities for the patient. A feature representation for a modality refers to a collection of features that collectively represent data from the modality. For convenience, a set of (scalar) features representing multi-modal data can be understood as being indexed by a set of dimensions, referred to as “feature” dimensions.

A few examples of possible modalities, and feature representations for these modalities, are described in more detail next.

In some implementations, multi-modal data characterizing a patient includes data derived from functional magnetic resonance imaging (fMRI) of the brain of the patient. fMRI data can be derived from a sequence of fMRI images, where each fMRI image corresponds to a respective time point in a sequence of time points and characterizes blood flow in the brain at the time point. More specifically, each fMRI image can be represented as array of voxels, where each voxel is associated with an intensity value that represents blood flow at a corresponding location in the brain.

To generate a feature representation of fMRI data of the brain of the patient, the machine learning system 100 can process the fMRI images to generate a respective blood flow curve for each brain region in a set of brain regions that collectively define a parcellation (i.e., partition) of the brain. The blood flow curve for a brain region can define, for each time point in the sequence of time points, the average blood flow in the brain region at the time point. The machine learning system 100 can compute the average blood flow in a brain region at a time point, e.g., by averaging the intensity values of the voxels in the brain region in the fMRI image for the time point. The machine learning system 100 can process the blood flow curves for the brain regions to generate an N×N “functional connectivity” matrix, where N is the number of regions in the parcellation, and where entry (i, j) of the functional connectivity matrix represents a correlation between the blood flow curves for brain region i and brain region j.

A few example techniques for deriving a feature representation of the fMRI data from the functional connectivity matrix are described in more detail next.

In one example, a feature representation of the fMRI data includes the functional connectivity matrix.

In another example, the machine learning system 100 can generate a feature representation of the fMRI data by projecting the functional connectivity matrix onto a vector, where each component of the vector is a combination (e.g., sum or average) of a respective row or column of the functional connectivity matrix.

In another example, to generate a feature representation of the fMRI data, the machine learning system 100 can process the functional connectivity matrix to generate an adjacency matrix that represents a graph. The machine learning system 100 can generate the adjacency matrix, e.g., by setting each value in the functional connectivity matrix exceeds a predefined threshold to 1, and setting each other value in the functional connectivity matrix to 0. The adjacency matrix represents a graph that includes: (i) a set of nodes, where each node corresponds to a respective brain region, and (ii) a set of edges, where each edge connects a respective pair of nodes in the graph. The adjacency matrix defines which nodes in the graph are connected by edges. In particular, an edge connects node i to node j if the value of entry (i, j) in the adjacency matrix of the graph is 1.

After generating the adjacency matrix representing the graph, the machine learning system 100 can generate a set of graph statistics characterizing the topology of the graph represented by the adjacency matrix, and the set of graph statistics can define the feature representation of the fMRI data. The machine learning system 100 can generate any appropriate graph statistics characterizing the topology of the graph represented by the adjacency matrix, e.g., an average measure of centrality (e.g., degree centrality, or PageRank centrality) of the nodes in the graph, an average size of connected components of the graph (where the size of a connected component of the graph can refer to, e.g., the number of nodes in the connected component of the graph), a diameter of the graph, etc.

In another example, to generate the feature representation of the fMRI data, the machine learning system 100 can instantiate a graph that includes: (i) a set of nodes, where each node corresponds to a respective brain region, and (ii) a set of edges, where each edge connects a respective pair of nodes in the graph. The graph can be a fully-connected graph, i.e., such that every pair of nodes in the graph is connected by a respective edge in the graph. The machine learning system 100 can further instantiate a respective node embedding for each node in the graph and a respective edge embedding for each edge in the graph. The node embedding for a node can be an embedding (e.g., a one-hot embedding) that identifies the brain region represented by the node. The edge embedding for an edge connecting a pair of nodes representing brain regions indexed by i and j can be an embedding representing the value of entry (i, j) in the functional connectivity matrix. Thus the machine learning system 100 can instantiate the edge embeddings for the edges in the graph using the functional connectivity matrix.

After instantiating the graph, the machine learning system 100 can process data defining the graph (including the node embeddings and the edge embeddings associated with the graph) using a graph neural network to generate a latent representation of the graph that defines the feature representation of the fMRI data. More specifically, at each of one or more time steps, the graph neural network can update the respective node embedding for each node in the graph by processing the current node embeddings and the current edge embeddings in accordance with values of a set of graph neural network parameters. The machine learning system 100 can then combine (e.g., sum or average) the updated node embeddings associated with the nodes in the graph as of the final time step to generate the feature representation of the fMRI data. The graph neural network can have any appropriate graph neural network architecture that enables it to perform its described function. Examples of graph neural network architectures are described with reference to: J. Zhou et al., “Graph neural networks: a review of methods and applications,” AI Open, Volume 1, 2020, pages 57-81.

Optionally, in addition to generating a “full” functional connectivity matrix representing functional connectivity between each pair of regions in the set of regions defining the parcellation of the brain, the machine learning system 100 can generate one or more “reduced” functional connectivity matrices. Each reduced functional connectivity matrix represents functional connectivity between each pair of regions in a respective proper subset of the set of regions in the parcellation of the brain. That is, each reduced functional connectivity matrix can be represented by an n×n matrix, where n is the number of regions in the corresponding proper subset of the set of regions in the parcellation of the brain, and entry (i, j) of the reduced functional connectivity matrix represents a correlation between the blood flow curves for brain region i and brain region j.

In some cases, the machine learning system 100 generates one or more reduced functional connectivity matrices that each represent functional connectivity between a respective set of brain regions that are involved in performing a respective biological function in the brain. Examples of biological functions include, e.g., visual data processing, auditory data processing, natural language processing, motor control, etc.

In some cases, the machine learning system 100 generates one or more reduced functional connectivity matrices that each represent functional connectivity between a respective set of brain regions that are anatomically connected in the brain, e.g., that are physically adjacent to one another in the brain.

The machine learning system 100 can generate a respective feature representation of each reduced functional connectivity matrix using any appropriate technique, including any of the techniques described above for generating a feature representation of a full functional connectivity matrix.

In some implementations, multi-modal data characterizing a patient can include clinical scale data obtained from a clinical interview with the patient. Clinical scale data for a patient includes a respective score for the patient in each of multiple categories, where each category is associated with a predefined set of possible scores (e.g., integer values between 1 and 10). Examples of possible categories include, e.g.: apparent sadness, reported sadness, inner tension, reduced sleep, reduced appetite, irritability, aggressiveness, etc. Examples of clinical scales include, e.g.: Positive and Negative Syndrome Scale (PANSS), Brief Assessment of Cognition in Schizophrenia (BACS), Young Mania Rating Scale (YMRS), and Montgomery-Asberg Depression Rating Scale (MADRS). The machine learning system 100 can generate a feature representation of clinical scale data, e.g., e.g., as a sequence of embeddings (e.g., one-hot embeddings), where each embedding represents the score for the patient in a respective category.

In some implementations, multi-modal data characterizing a patient includes electroencephalography (EEG) data. Generally, EEG data includes a respective voltage waveform measured by each of one or more electrodes that are placed at respective locations in proximity to the brain of the patient. The voltage waveform measured by an electrode includes a respective voltage measurement at the location of the electrode at each time point in a sequence of time points.

The machine learning system 100 can generate a feature representation of EEG data in a variety of possible ways. For example, the machine learning system 100 can generate a feature representation of the EEG data by stacking each of the voltage waveforms into a waveform array, e.g., such that each row or column of the waveform array represents a respective voltage waveform. As another example, the machine learning system 100 can transform each voltage waveform into a different domain, e.g., by applying a Fourier transform to each voltage waveform to transform the voltage waveform into a frequency domain, and then stack the transformed voltage waveforms into a transformed waveform array.

In some implementations, multi-modal data characterizing a patient includes genomic data. The machine learning system 100 can represent genomic data in any of a variety of possible formats. A few examples techniques for representing genomic data are described in more detail next.

In one example, the machine learning system 100 can represent genomic data as a sequence of nucleotides from the genome of the patient, where each nucleotide includes a respective nucleobase from a set of possible nucleobases (in particular: guanine, adenine, cytosine, and thymine). The machine learning system 100 can generate a feature representation of the genomic data, e.g., as a sequence of embeddings, where each embedding corresponding to a respective nucleotide in the sequence of nucleotides and identifies the nucleobase included in the nucleotide.

In another implementation, the machine learning system 100 can represent genomic data with reference to a predefined set of genes. In particular, the machine learning system 100 can measure a respective degree to which each gene in the predefined set of genes is expressed in the genome of the patient, and the collection of gene expression values can collectively define the genomic data.

In another example, the machine learning system 100 can represent genomic data with reference to a predefined set of locations of interest in the genome of the patient. In particular, the machine learning system 100 can generate a respective representation (e.g., one-hot embedding) identifying the nucleobase included in the nucleotide at each location of interest in the genome of the patient. The representations of the nucleobases at the locations of interest in the genome of the patient can collectively define the genomic data.

In some implementations, multi-modal data characterizing a patient includes proteomic data, e.g., that characterizes the expression levels of various proteins in the patient. The proteomic data represent, for each protein in a predefined set of proteins, a level of expression of the protein in the patient.

In some implementations, multi-modal data characterizing a patient includes audio data, e.g., that represents a sequence of words spoken by the patient. The feature representation of the audio data can include, e.g., an audio waveform that includes a respective audio sample at each time point in a sequence of time points, or a spectrogram representation.

In some implementations, multi-modal data characterizing a patient includes video data that shows, e.g., the face of the patient or the entire body of the patient as the patient performs a task, e.g., speaking a sequence of words. The video data can be represented, e.g., as a sequence of video frames, or as a sequence of facial activity vectors. Each facial activity vector can correspond to a respective video frame, and can identify whether the face of the patient in the corresponding video frame is exhibiting each facial activity in a set of possible facial activities, e.g., eyes downcast, eyes turned left, eyes turned right, eyebrows raised, etc.

In some cases, multi-modal data characterizing a patient can include multiple feature representations for certain modalities in the set of modalities (i.e., rather than only a single feature representation for each modality). For example, the multi-modal data can include multiple feature representations corresponding to the fMRI modality, including respective feature representations of a full functional connectivity matrix and one or more reduced functional connectivity matrices, as described above.

The machine learning system 100 includes a patient clustering system 200, an encoder training system 300, a patient classification system 310, and a classifier training system 320, which will each be described in more detail below.

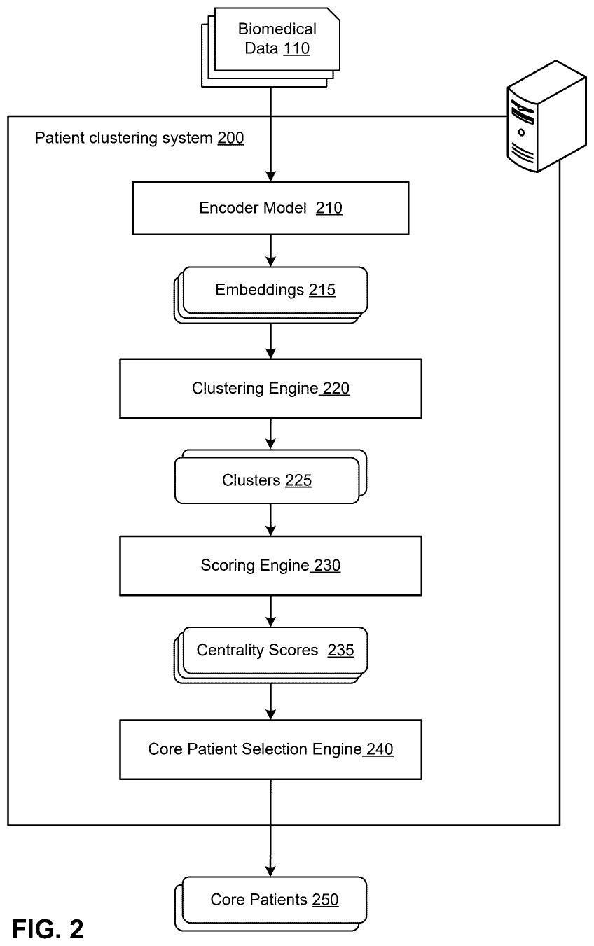

The patient clustering system 200 is configured to group the patients characterized by the biomedical data 110 into a set of patient clusters. An example of a patient clustering system 200 is described in more detail below with reference to FIG. 2.

In general, each of the patient clusters can represent a patient category, and the clusters define a partition of the population of patients into patient categories. In some cases, the clustering is performed on a set of embeddings (generated by an encoder machine learning model 210) in a latent space representing respective multi-modal data for each patient in the population of patients.

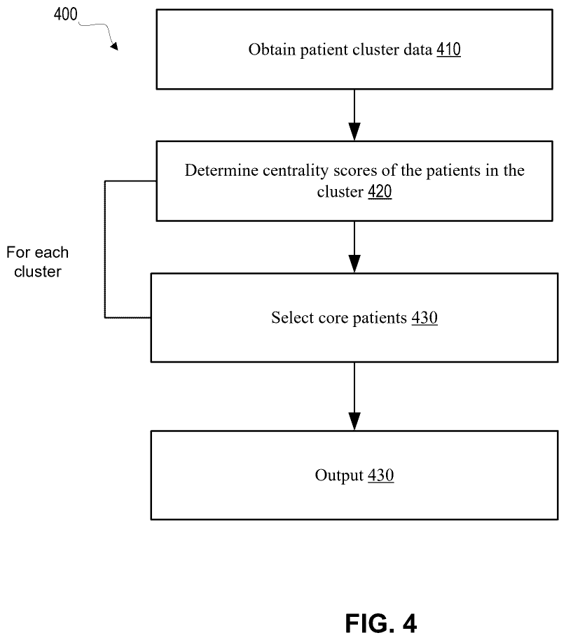

The patient clustering system 200 is further configured to identify, for each respective cluster in the set of clusters, a respective subset of patients included in the respective cluster as core patients for the respective cluster. In some cases, the core patients in each cluster are a proper subset of the patients included in the cluster. That is, not all patients in the cluster are identified as core patients for the cluster. As described in further detail with reference to FIG. 2 and FIG. 4, the core patients for each patient cluster can be identified based on the centrality scores of the patients that characterize how closely each patient is associated with the patient cluster.

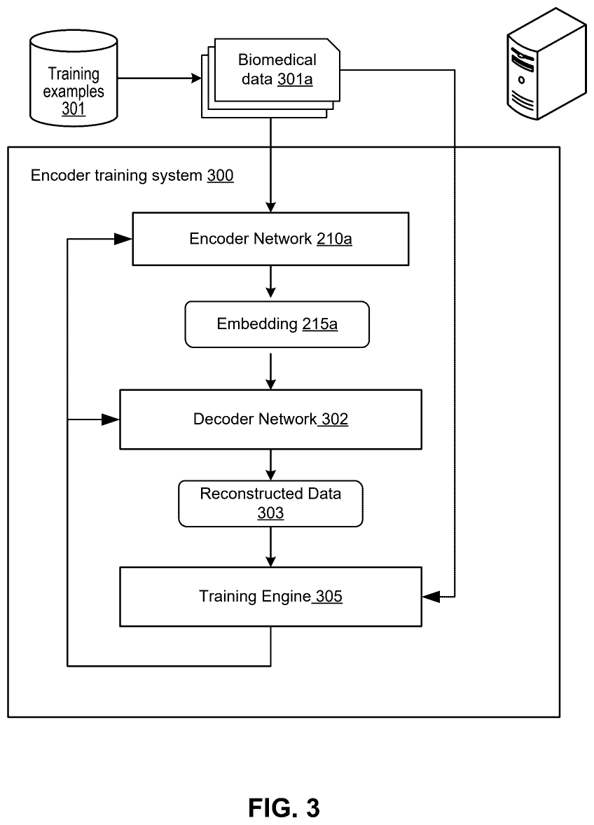

The encoder training system 300 is configured to train the encoder machine learning model 210, i.e., determine the model parameters of the encoder machine learning model 210. An example of the encoder training system 300 is described in more detail below with reference to FIG. 3.

The machine learning system 100 can further include a classifier training system 320 configured to train a classification machine learning model 315 using the core patients 250 identified by the patient clustering system 200. The classification machine learning model 315 is configured to process an input characterizing the biomedical data 312 of a particular patient to generate an output that characterizes a classification of the patient, e.g., which of the set of clusters (categories or classes) the particular patient belongs to.

In some implementations, the output of the model 315 can specify a classification label that corresponds to the predicted outcome for the input features. For example, the output can be a specific disease category, a predicted positive or negative diagnosis for a particular disease, a predicted outcome category for a particular treatment, or a risk level of serious side effects of a particular treatment. In some other implementations, the output of the model 315 can specify a respective classification score for each patient cluster (category) that represents a likelihood that the particular patient is included in the patient cluster. The classification machine learning model 315 can be any appropriate machine learning model, e.g., a neural network model, a decision tree, or a Support Vector Machine (SVM).

The classifier training system 320 generates a set of training examples based on only core patients in the population of patients, where each training example corresponds to a core patient from the population of patients. Each training example includes (i) a training input that includes the set of biomedical data characterizing the core patient, and (ii) a target output that includes a label that identifies the patient cluster that includes the core patient.

Training the classification model 315 exclusively on the core patients 250 offers several advantages. By limiting the training examples to core patients instead of the entire patient population, the system reduces the number of training examples required, leading to faster training times and lower computational demands. This approach also helps prevent overfitting, where the training is negatively impacted by noises or idiosyncrasies in the data of the broader patient population that do not generalize well to new data. Furthermore, the core patients 250, which are selected to represent the characteristics of their respective clusters, provide a more generalized representation of the overall patient population within that cluster. Training with the core patient data allows the model to learn patterns that are more likely to generalize well to unseen patients belonging to the same cluster, resulting in more accurate classification. Essentially, by using training data comprising smaller, more representative groups, the system 315 achieves more efficient training and potentially improves the accuracy of classification results.

The system 320 trains the classification machine learning model on the set of training examples. In some implementations, the classification machine learning model 315 includes a neural network. The system 320 can update the parameters of the neural network over one or more training iterations. In each training iteration, the system 320 uses the neural network, according to the current values of the parameters of the neural network, to process one or more training inputs from a batch of one or more training examples to generate one or more training outputs that predict the classifications for the training inputs. The system 320 can update the values of the parameters to optimize a loss function. The loss function includes a classification loss that measures the differences between the training outputs and the target output labels. The classification loss can take any appropriate form, e.g., as a cross-entropy loss. The system 320 can optimize the loss function using any appropriate machine learning training technique, e.g., stochastic gradient descent.

In some implementations, the classification machine learning model 315 is a decision tree model that includes internal nodes, branches, and leave nodes. Each internal node in the tree represents a decision based on a specific feature, where a test condition is applied to the feature values to split the data into subsets. These nodes have branches leading to child nodes, which can be either additional decision nodes or terminal leaf nodes. Leaf nodes represent the final outcome or class label.

To train the decision tree model, the system 320 recursively partitions the training examples according to a respective feature at each internal node. At the root node of the decision tree, the system can evaluate all available features to determine the best feature and the corresponding condition (e.g., a numerical threshold) that maximally splits the training examples into subsets that are more homogeneous in terms of their target outputs (patient clusters). This evaluation can utilize any appropriate metrics, such as information gain, Gini impurity, or entropy to quantify the effectiveness of each split. Once a feature and condition are chosen for the root node, the training examples are partitioned into two or more subsets based on the selected feature. This partitioning process is repeated recursively for each subset at each subsequent decision node, where the system selects the next best feature and condition to further split the data. The hierarchical structure of the decision tree emerges as these splits continue until a stopping criterion is met, such as reaching a maximum tree depth, minimum samples per leaf, or no further significant improvement in impurity reduction. By recursively partitioning the data based on features and optimizing split criteria, the system 320 constructs a hierarchical set of decision rules that map input biomedical data to predicted patient clusters.

The machine learning system further includes a patient classification system 310 configured to use the trained classification machine learning model 315 to generate a classification output 330 for a new patient based on the biomedical data of the biomedical data 312 of the new patient. As described above, the classification machine learning model 315 is configured to process an input characterizing the biomedical data 312 of the new patient to generate an output that characterizes a classification of the patient, e.g., specifies which of the set of clusters (categories or classes) the particular patient belongs to. In some cases, the classification output 330 can be used to generate a recommendation 340 for a clinical treatment of the new patient. In some cases, the classification output 330 can be used to determine administering a drug to the new patient.

In one example, the set of classes can include one class for patients that are classified as having responded to a medical treatment, and another class for patients that are classified as having not responded to the medical treatment. In this example, the classification output 330 can provide an indication of whether the new patient is likely to respond to the medical treatment. Based at least in part on the predicted indication that the patient will respond to the medical treatment, a human expert (e.g., a physician) can determine whether the medical treatment should be applied to the patient, and in some cases, proceed to apply the medical treatment to the patient. Applying a medical treatment to a patient can include administering a drug to the patient.

In another example, the set of classes can include one class for patients that have been classified as having experienced significant side effects from receiving a medical treatment, and another class for patients that are classified as having not experienced significant side effects from receiving the medical treatment. In this example, the classification output 330 can provide an indication of whether the new patient is likely to experience significant side effects from receiving the medical treatment. Based at least in part on the predicted indication that the patient will experience significant side effects from the medical treatment, a human expert (e.g., a physician) can determine whether the medical treatment should be applied to the patient, and in some cases, proceed to apply the medical treatment to the patient.

In another example, the set of classes can include one class for patients that have been diagnosed with a medical condition, and a second class for patients that have not been diagnosed with the medical condition. In this example, the classification output 330 can provide an indication of whether the new patient is likely to have the medical condition. Based at least in part on the predicted indication that the patient has the medical condition, a human expert (e.g., a physician) can determine whether the patient should be diagnosed with the medical condition.

As described above, the biomedical data 312 of the new patient can include multi-modal data that includes a respective feature representation for each modality in a set of multiple modalities for the patient. In some implementations, the classification machine learning model 315 is trained subject to a constraint that classifications generated by the classification machine learning model 315 depend on at most a predefined, maximum number of biomedical features, e.g., 2, 3, 4, or 5 features. In other words, the model 315 only uses a predefined maximum number of features to classify patients into different clusters. The focus on a limited set of features offers several advantages. For example, by relying on fewer features, the output generated by the model 315 is more interpretable. Clinicians or researchers can more readily identify which features are most critical for distinguishing between the patient clusters. Furthermore, training and using a model with fewer features can be computationally less expensive and faster compared to models that utilize a large number of features. This translates to quicker analysis times and potentially lower resource requirements.

In some cases, the classification machine learning model 315 is a decision tree model, and the constraint is defined as the maximum depth for the decision tree. As described above, a decision tree is a hierarchical model that includes internal nodes, branches, and leave nodes. Each internal node in the tree represents a decision based on a specific feature, where a test condition is applied to the feature values to split the data into subsets. These nodes have branches leading to child nodes, which can be either additional decision nodes or terminal leaf nodes. Leaf nodes represent the final outcome or class label.

Once the decision tree classification model 315 is trained, the system 310 can use it to process the multi-modal biomedical data 312 of the new patient to reach a classification by following a series of hierarchical decisions from the root node to a leaf node of the decision tree. For a given patient, the decision tree evaluates various biomedical features sequentially, starting from the root node. As described above, these features in the multi-modal data can include features or representations of imaging data, clinical interview records, clinical and laboratory test data, genomic data, audio data, and/or video data obtained for the patient. At each decision node, the decision tree model applies a specific condition or test based on one of these features. The outcome of this test directs the input data point to one of the node's branches, leading to the next node, which may be another decision node or a leaf node. Each decision node splits the data based on the feature's value, effectively narrowing down the potential classifications as the patient data moves deeper into the tree.

This process continues until a leaf node is reached, representing the final decision for the classification. The leaf node contains the predicted classification for the patient, such as a diagnosis or a risk category. The decision path through the tree represents a logical sequence of decisions based on the patient's feature values, ultimately leading to the classification that the model predicts. This method is inherently interpretable, as the path taken by the patient data can be easily traced, revealing which biomedical features and thresholds were critical in determining the classification. This transparency makes decision trees a valuable tool not only for predictive accuracy but also for understanding and explaining the decision-making process in clinical settings, helping healthcare providers make informed decisions based on a comprehensive analysis of multi-modal biomedical data.

Limiting the maximum number of biomedical features processed by the decision tree, which corresponds to the maximum tree depth, can help prevent overfitting of the model during training. A decision tree with limited depth captures the most significant patterns in the biomedical data without becoming overly complex, which is particularly important in a clinical setting where interpretability and robustness are crucial. By focusing on the most relevant features and reducing the risk of modeling noise or minor variations in the training data, a decision tree with limited depth provides more reliable and clinically meaningful predictions. Additionally, simpler trees are easier for healthcare providers to understand and trust, facilitating their integration into clinical decision-making processes and enhancing their usability in practice. Furthermore, training and using a decision tree with reduced depth is computationally more efficient, leading to quicker analysis times and potentially lower resource requirements.

FIG. 2 shows an example patient clustering system 200. The patient clustering system 200 is an example of a system implemented as computer programs on one or more computers in one or more locations in which the systems, components, and techniques described below are implemented.

As described above with reference to FIG. 1, the patient clustering system 200 is configured to group a population of patients characterized by the biomedical data 110 into a set of patient clusters, and identify, for each respective cluster in the set of clusters, a respective subset of patients included in the respective cluster as core patients 250 for the respective cluster.

As described above with reference to FIG. 1, in some implementations, the biomedical data 110 includes, for each patient, multi-modal data that includes a respective feature representation for each modality in a set of multiple modalities for the patient. The multi-modal data can include, e.g., features or representations of imaging data, clinical interview records, clinical test data, genomic data, audio data, and/or video data obtained for each patient.

The patient clustering system 200 includes an encoder machine learning model 210, a clustering engine 220, a scoring engine 230, and a core patient selection engine 240.

The encoder machine learning model 210 is configured to process, for each patient, an input specifying the set of biomedical data characterizing the patient to generate a biomedical data embedding 215 of the set of biomedical data in a latent space. In a particular example, the encoder machine learning model 210 receives input multi-modal biomedical data that includes multiple modality feature representations. Each modality feature representation includes a collection of features that collectively represent data from a corresponding modality. Examples of modality feature representations are described with reference to FIG. 1.

In general, the embedding 215 generated by the encoder machine learning model 210 has a lower dimensionality than the biomedical data 110 itself, and thus the embedding 215 provides a compressed representation of the biomedical data 110. The embeddings 215 generated by the encoder machine learning model 210 enable more efficient use of computational resources during processing of the biomedical data 110. In particular, the embeddings 215 occupy less space than the original data when stored in a memory, and downstream processing of the embeddings requires fewer arithmetic operations (e.g., additions and multiplications) than would be required to process the original data.

The encoder machine learning model 210 can be any appropriate machine learning model. For example, the encoder machine learning model 210 can include an encoder neural network. As described in further detail with reference to FIG. 3, before using the encoder neural network to generate the embeddings 215, an encoder training system can train the encoder neural network jointly with a decoder neural network.

In a particular example, the encoder neural network can include multiple encoder subnetworks, where each encoder subnetwork corresponds to a respective modality and is configured to receive as input a feature representation of the corresponding modality. Each encoder subnetwork can process a corresponding modality feature representation to generate a respective subnetwork output, e.g., a respective set of parameters that define a probability distribution over the latent space. For example, each encoder subnetwork Eᵢ can generate a mean vector μᵢ and a covariance matrix Vᵢ of a Normal distribution over the latent space. The encoder neural network can combine the subnetwork outputs to generate a combined output, e.g., parameters of a “posterior” probability distribution over the latent space. For example, if each encoder subnetwork generates mean and covariance parameters of a Normal distribution, as described above, then the encoder neural network can generate the a mean vector μ and a covariance matrix V of the posterior probability distribution as:

$$μ = ( {\sum }_{i = 0 … n} {μ}_{i} {V}_{i}^{- 1} ) {( {\sum }_{i} {V}_{i}^{- 1} )}^{- 1} \qquad ( 1 )$$

$$V = {( {\sum }_{i = 0 … n} {V}_{i} )}^{- 1} \qquad ( 2 )$$

where μ₀ is a mean vector of a predefined “prior” Normal probability distribution, V₀ is a covariance matrix of the predefined prior Normal distribution, and for each i∈{1, . . . , n}, μᵢ is the mean vector generated by encoder subnetwork i and Vᵢ is the covariance matrix generated by encoder subnetwork i. The encoder neural network can generate the embedding 215 of the input multi-modal data using the posterior probability distribution over the latent space. For example, the encoder neural network can select the embedding of the input multi-modal data as the mean of the posterior probability distribution over the latent space.

In some cases, the input multi-modal data can be incomplete, i.e., certain modality feature representations can be missing from the input data. This can occur, e.g., if data from certain modalities were not collected for a patient, or are otherwise unavailable for a patient. In this situation, the encoder neural network can generate the embedding 215 by processing the available modality feature representations using the corresponding encoder subnetworks, and combining the outputs of the encoder subnetworks in accordance with equations (1)-(2). Encoder subnetworks that are configured to process the missing modality feature representations are not used to generate the embedding 215.

Generally, each of the encoder subnetworks can have any appropriate neural network architecture which enables them to perform their described functions. In particular, each encoder subnetwork can have any appropriate types of neural network layers (e.g., fully connected layers, convolutional layers, attention layers, etc.) in any appropriate numbers (e.g., 5 layers, 25 layers, or 50 layers) and connected in any appropriate configuration (e.g., as a linear sequence of layers).

The clustering engine 220 is configured to cluster the patients in the population of patients, based on the respective biomedical data embedding 215 associated with each patient, to identify a set of patient clusters 225.

Generally, the clustering engine 220 performs a clustering operation that encourages the embeddings in the same cluster to be more similar (accordingly to some similarity measure in the latent space) than embeddings in different clusters. The clustering engine 220 can cluster the embeddings 215 using any appropriate clustering operation, e.g., a k-means clustering operation, an expectation maximization clustering operation, a hierarchical agglomerative clustering operation, or a spectral clustering operation. The numbers of clusters generated by the clustering engine 220 can be, e.g., a predefined hyper-parameter that is specified by a user of the patient clustering system, or determined dynamically by the clustering engine 220 during clustering.

In some implementations, prior to performing the clustering operation on the embeddings 215, the patient clustering system 200 can apply a projection operation to each embedding 215 to remove one or more specified dimensions of the embedding 215. Thus, in these implementations, the clustering engine 220 clusters projected embeddings having fewer dimensions than the original embeddings 215 generated by the encoder model 210. The dimensions to be removed from the embeddings 215 can be specified, e.g., by a user of the patient clustering system 200 or by another system.

The patient clustering system 200 identifies each cluster 225 of embeddings 215 generated by the clustering engine 220 as representing a respective patient category. The patient clustering system 200 further identifies each patient in the population of patients as being included in the patient category represented by the cluster that includes the embedding of the multi-modal data characterizing the patient.

The scoring engine 230 is configured to determine a centrality score 235 for each patient included in each patient cluster. The centrality score 235 characterizes how closely the patient is associated with the patient cluster based on their biomedical data embedding 215. The core patient selection engine 240 is configured to select a subset of patients within each patient cluster as core patients 250, based on the centrality scores 235. For example, the core patient selection engine 240 can select patients with centrality scores 235 above a predefined threshold as the core patients 250 for each patient cluster 225. In another example, the core patient selection engine 240 can select a predefined number of patients with the highest centrality scores 235 as the core patients 250.

The scoring engine 230 can determine the centrality scores 235 using any of a variety of techniques.

In some cases, the patient clustering system 200 can determine, for each patient, a measure of stability regarding the assignment of the patient to the patient cluster that includes them over multiple instances of clustering. The scoring engine 230 can then determine the centrality score for each patient based, at least in part, on the stability of their cluster assignment. To determine the stability measure, the system 200 can use the clustering engine 220 to perform multiple instances of the clustering process, with each instance generating a different set of patient clusters. The system 200 can assess stability by measuring the consistency of the patient's inclusion in the same patent cluster across these multiple clustering instances.

Specifically, the system 200 can determine the measure of stability for each patient by evaluating the degree of overlap between the clusters that include the patient across the different clustering instances. A higher overlap indicates that the patient's assignment to a particular cluster is consistent and stable, contributing to a higher centrality score. This stability measure can indicate confidence in the clustering results, ensuring that the patients with high centrality scores (i.e., the core patients) are robustly categorized, and that the resulting clusters of core patients are reliable and meaningful for subsequent analysis and decision-making.

In some cases, the scoring engine 230 can determine the centrality score by leveraging machine learning techniques. For instance, the scoring engine 230 can use a confidence measure that characterizes a discriminative machine learning model's confidence in classifying a patient into a specific patient cluster. The system 200, or another system, can train the discriminative machine learning model to process patient data and generate a discriminative output that classifies the patient into one of the predefined patient clusters from the set of patient clusters. The discriminative machine learning model can be trained on labeled data, where each patient is assigned to a specific cluster, enabling the model to learn the distinguishing features of each cluster.

The discriminative model can be a neural network, support vector machine, or another classifier, that maps input features to cluster labels. Once trained, the discriminative model can output a respective probability score or confidence level for each category (cluster), indicating the likelihood that the patient belongs to a particular cluster. The scoring engine 230 can determine the confidence measure from the output of the discriminative model and use the confidence measure to determine the centrality score, with higher confidence in the classification leading to higher centrality scores. This approach ensures that patients with higher centrality scores (i.e., the core patients) are those whose inclusion in their respective clusters is most certain.

In some cases, the scoring engine 230 can determine the centrality score using the distribution of biomedical data embeddings 215 within each patient cluster 225. The scoring engine 230 can determine a distribution function for each patient cluster 225 that characterizes the distribution of biomedical data embeddings of the patients within that cluster. This distribution function can be a statistical representation, such as a Gaussian distribution or another appropriate probabilistic model, that captures the distribution of the embeddings within the cluster. This distribution function for a particular cluster can be understood as describing the probability of finding the embeddings within different regions of the latent space in that cluster.

Once the distribution function for each patient cluster is established, the scoring engine 230 can evaluate a likelihood for each patient within their assigned cluster using the distribution function. The scoring engine 230 can determine the centrality score for each patient based at least on the likelihood evaluated using the distribution function. A higher centrality score indicates that the patient's embedding is closer to the central tendency of the cluster. Thus, by using this approach, the patients with higher centrality scores (i.e., the core patients) are selected as those whose data characteristics align closely with the core attributes of their cluster.

In some cases, to determine the centrality score, the scoring engine 230 can utilize the concept of centroids within each patient cluster 225. The process begins by calculating a centroid for each patient cluster, which represents the average or central point of the biomedical data embeddings of all patients within that cluster 225. This centroid serves as a reference point that characterizes the central tendency of the cluster's biomedical data.

Once the centroids are determined, the scoring engine 230 can determine the centrality score for each patient based on the distance between the patient's biomedical data embedding and the centroid of their respective cluster. A shorter distance indicates that the patient's embedding is closer to the central characteristics of the cluster, thus resulting in a higher centrality score. Conversely, patients whose embeddings are farther from the centroid result in lower centrality scores, reflecting a weaker association with the central characteristics of the cluster. This approach provides a straightforward and interpretable way to assess how representative each patient is within their cluster. By focusing on the distance to the centroid, the scoring engine 230 ensures that patients are more representative of the cluster's central attributes are assigned higher centrality scores, and are more likely to be selected as the core patents 250.

In some cases, the scoring engine 230 can combine two or more of the techniques described above to determine the centrality scores. For example, the scoring engine 230 can compute a combined centrality score using two or more centrality scores that have been computed using two or more different techniques described above, e.g., by using a weighted sum.

FIG. 3 shows an example encoder training system 300. The encoder training system 300 is an example of a system implemented as computer programs on one or more computers in one or more locations in which the systems, components, and techniques described below are implemented. As discussed above, the encoder machine learning model 210 can include an encoder neural network, e.g., the encoder neural network 210a shown in FIG. 3.

The encoder training system 300 can jointly train the encoder neural network 210a and a decoder neural network 302 on a set of training examples 301. Each training example 301 corresponds to a respective patient and can include biomedical data 301a characterizing the patient. In some cases, the biomedical data 301a can include multi-modal data as described in further detail with reference to FIG. 1.

To jointly train the encoder neural network 210a and the decoder neural network 302 on a training example, the encoder training system 300 processes the multi-modal data from the training example using the encoder neural network 210a, in accordance with values of a set of encoder neural network parameters, to generate an embedding 215a of the biomedical data 301a. The training system 300 then processes the embedding 215a using the decoder neural network 302, in accordance with values of a set of decoder neural network parameters, to generate reconstructed biomedical data 303 that defines a reconstruction (i.e., an estimate) of the biomedical data 301a from the training example 301.

The decoder neural network 302 can have any appropriate model architecture. In a particular example, the decoder neural network 302 can include multiple decoder subnetworks. Each decoder subnetwork is configured to process an embedding from the latent space (e.g., an embedding generated by the encoder neural network) to generate a corresponding modality feature representation. The collection of modality feature representations generated by the decoder subnetworks collectively define the reconstructed data 303. Each of the decoder subnetworks can have any appropriate neural network architecture which enables them to perform their described functions. In particular, each decoder subnetwork can have any appropriate types of neural network layers (e.g., fully connected layers, convolutional layers, attention layers, etc.) in any appropriate numbers (e.g., 5 layers, 25 layers, or 50 layers) and connected in any appropriate configuration (e.g., as a linear sequence of layers).

The training system 300 includes a training engine 305 configured to update the respective values of the encoder neural network parameters and the decoder neural network parameters to optimize an objective function that includes a reconstruction error term. The reconstruction error term measures an error between: (i) the biomedical data 301a from the training example, and (ii) the reconstruction 303 of the biomedical data from the training example. The training engine 305 can optimize the objective function using any appropriate machine learning training technique, e.g., stochastic gradient descent.

The training encourages the encoder neural network 210a to generate embeddings of biomedical data that preserve the information content of the biomedical data, i.e., such that the biomedical data can be reconstructed by the decoder neural network 302 by processing the embeddings.

After the encoder neural network 210a and the decoder neural network 302 have been jointly trained by the encoder training system 300, the encoder neural network 210a can be provided for use by the patient clustering system 200 (as described with reference to FIG. 2) to generate the embeddings 215 for biomedical data 110 of the population of patients. In some cases, the training examples 301 used by the encoder training system 300 are obtained from patients who are different from the population of patients to be clustered by the patient clustering system 200. In some other cases, the biomedical data 110 from one or more patients within the population to be clustered by the patient clustering system 200 can be included as part of the training examples 301 used by the encoder training system 300.

FIG. 4 is a flow diagram of an example process 400 for determining a subset of core patients for each of a set of patient clusters. For convenience, the process 400 will be described as being performed by a system of one or more computers located in one or more locations. For example, the patient clustering system, e.g., the patient clustering system 200 of FIG. 2, appropriately programmed in accordance with this specification, can perform the process 400.

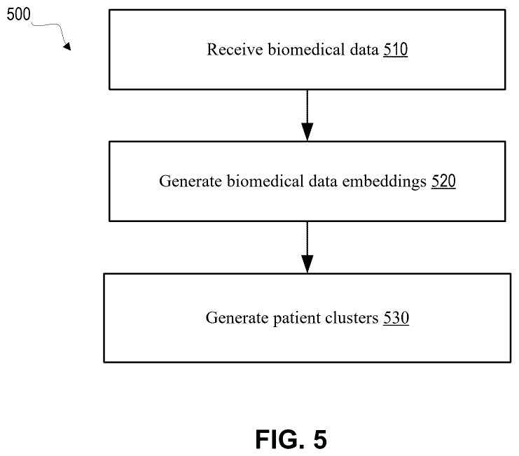

At 410, the system obtains patient cluster data that defines a set of patient clusters. The patient cluster data includes, for each of the set of patient clusters, a biomedical data embedding associated with each patient that's included in the patient cluster. In some cases, the patient cluster data can be obtained by generating biomedical data embeddings for a population of patients and subsequently clustering the population based on these biomedical data embeddings. These operations are described in further detail with reference to FIG. 2 and FIG. 5.

After obtaining the patient cluster data, the system selects, for each patient cluster in the set of patient clusters, a proper subset of a plurality of patients included in the patient cluster as core patients for the patient cluster. In particular, the system performs operations of 420 and 430 for each patient cluster in the set of patient clusters.

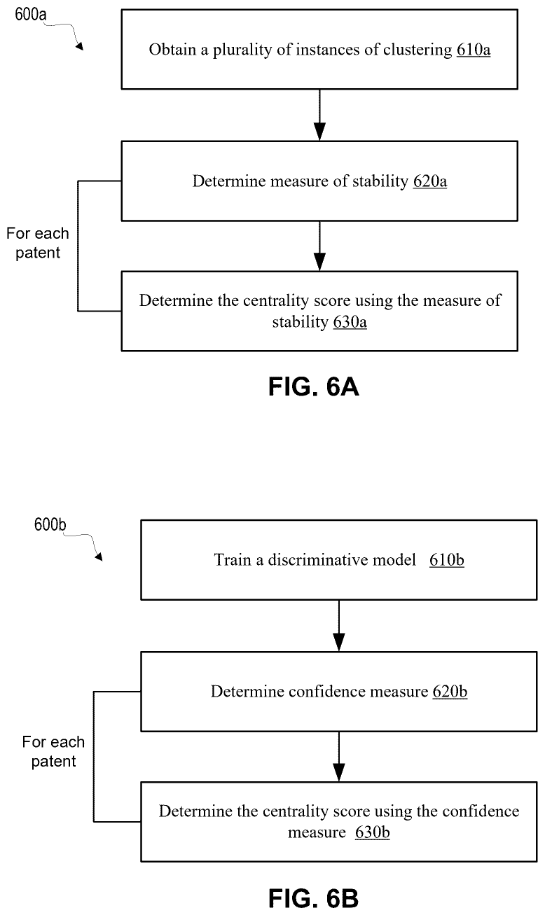

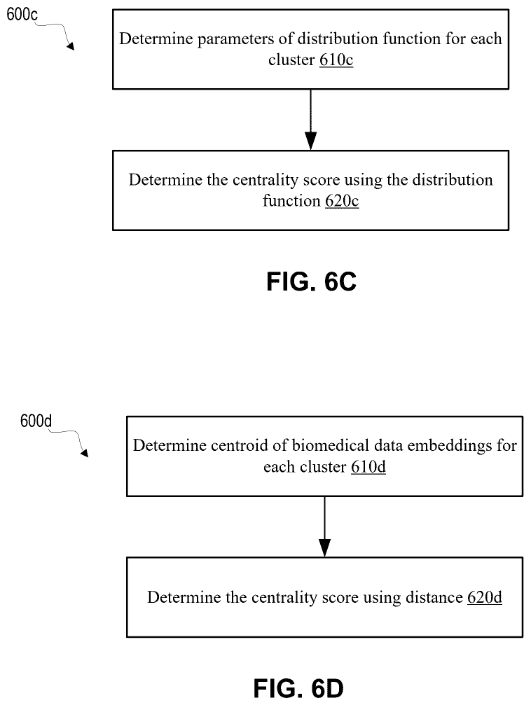

at 420, the system generates, for each patient included in the patient cluster, a centrality score characterizing how closely the patient is associated with the patient cluster based on the biomedical data embedding associated with the patient. The system can determine the centrality scores using any of a variety of techniques. Examples of techniques for determining the centrality scores are described with references to FIG. 2 and FIG. 6A-6D.

At 430, the system selects the proper subset of the plurality of patients included in the patient cluster as core patients for the patient cluster based on the centrality scores. The system can select the core patients based on the centrality scores using a number of different ways. For example, the system can select patients with centrality scores above a predefined threshold as the core patients for each patient cluster. In another example, the system can select a predefined number of patients with the highest centrality scores as the core patients for each patient cluster.

At 440, the system outputs data identifying: (i) the set of patient clusters, and (ii) the core patients for each patient cluster. The system or another system can use the output data in a number of different ways.

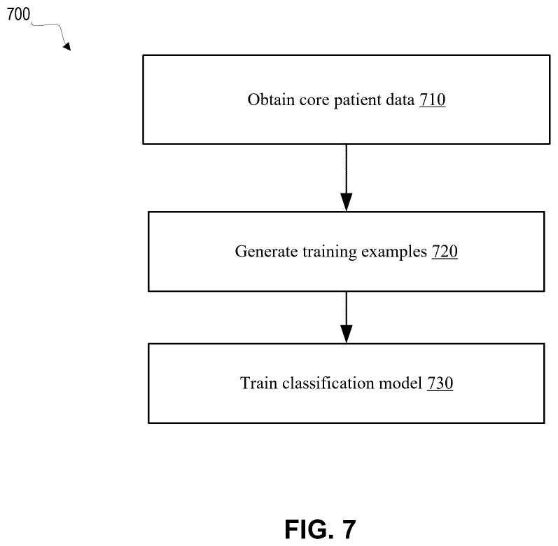

For example, the system or another system can use the output data to train a classification machine learning model using the core patients identified by the output data. The classification machine learning model is configured to process an input characterizing the biomedical data of a particular patient to generate an output that characterizes a classification of the patient, e.g., which of the set of clusters (categories or classes) the particular patient belongs to. Further details of training the classification machine learning model using the core patients are described with reference to FIG. 1 and FIG. 7.

In another example, the system or another system can determine, for each patient cluster, a set of statistics that characterize the patient cluster based on only the core patients of the patient cluster. The statistics can include central tendency measures (e.g., mean or median values), variability measures (e.g., standard deviation), and distribution analysis (e.g., histograms) of various biomedical data features. Additionally, the system can calculate the prevalence of specific conditions or treatments among core patients, identify common patterns or anomalies, and generate summary reports that highlight the defining characteristics of each cluster. The statistics computed based on the core patients can provide a more accurate and representative understanding of the cluster's characteristics, as the core patients are selected for their high centrality and representativeness of the cluster's core features. By focusing on core patients, the system can reduce the noise and variability introduced by outliers or less representative patients, leading to more robust and meaningful statistical insights.

FIG. 5 is a flow diagram of an example process 500 for determining patent clusters. For convenience, the process 500 will be described as being performed by a system of one or more computers located in one or more locations. For example, the patient clustering system, e.g., the patient clustering system 200 of FIG. 2, appropriately programmed in accordance with this specification, can perform the process 500.

At 510, the system receives, for each patient in a population of patients, a set of biomedical data characterizing the patient. As described with reference to FIG. 1 and FIG. 2, in some implementations, the biomedical data can include multi-modal data that includes a respective feature representation for each modality in a set of multiple modalities for the patient. The multi-modal data can include, e.g., features or representations of imaging data, clinical interview records, clinical test data, genomic data, audio data, and/or video data obtained for each patient.

At 520, the system processes, for each patient in the population of patients, the set of biomedical data characterizing the patient using an encoder machine learning model to generate a biomedical data embedding of the set of biomedical data in a latent space. Details of the encoder machine learning model and using the model to generate the biomedical data embeddings are described with reference to FIG. 2.

At 530, the system clusters the patients in the population of patients, based on the respective biomedical data embedding associated with each patient, to identify the set of patient clusters. Details of the process of clustering the patients are described with reference to FIG. 2.

FIG. 6A is a flow diagram of an example process 600a for determining patient centrality scores. For convenience, the process 600a will be described as being performed by a system of one or more computers located in one or more locations. For example, the patient clustering system, e.g., the patient clustering system 200 of FIG. 2, appropriately programmed in accordance with this specification, can perform the process 600a.

At 610a, the system obtains a plurality of instances of clustering. In particular, the system can perform the plurality of instances of the clustering, wherein each instance of the clustering generates a respective set of patient clusters.

At 620a, the system determines, for each patient, a measure of stability of an assignment of the patient to the patient cluster that includes the patient over a plurality of instances of clustering. For example, the system can determine, for each patient, the measure of stability based on a measure of overlap between the patient clusters that include the patient over the plurality of instances of the clustering.

At 630a, the system determines, for each patient in each patient cluster, the centrality score for the patient based at least in part on the stability of the assignment of the patient to the patient cluster that includes the patient.

Details of the process 600a are further described with reference to FIG. 2.

FIG. 6B is a flow diagram of another example process 600b for determining patient centrality scores. For convenience, the process 600b will be described as being performed by a system of one or more computers located in one or more locations. For example, the patient clustering system, e.g., the patient clustering system 200 of FIG. 2, appropriately programmed in accordance with this specification, can perform the process 600b.

At 610b, the system trains a discriminative machine learning model to process data characterizing a patient to generate a discriminative output that classifies the patient as being included in a respective one of the patient clusters from the set of patient clusters.

The system then generates the centrality score for each patient in each patient cluster. At 620b, the system determines a confidence measure that characterizes a confidence of the trained discriminative machine learning model in classifying the patient as being included in the patient cluster. At 630b, the system determines the centrality score for the patient based on at least on the confidence measure.

Details of the operations of process 600b are further described with reference to FIG. 2.

FIG. 6C is a flow diagram of an example process 600c for determining patient centrality scores. For convenience, the process 600c will be described as being performed by a system of one or more computers located in one or more locations. For example, the patient clustering system, e.g., the patient clustering system 200 of FIG. 2, appropriately programmed in accordance with this specification, can perform the process 600c.

At 610c, the system determines, for each patient cluster, parameters of a distribution function that characterizes a distribution of biomedical data embeddings of patients included in the patient cluster. At 620c, the system determines, for each patient in each patent cluster, the centrality score for the patient based at least in part on a likelihood of the biomedical data embedding of the patient under the distribution function for the patient cluster.

Details of the operations of process 600c are further described with reference to FIG. 2.

FIG. 6D is a flow diagram of an example process 600d for determining patient centrality scores. For convenience, the process 600d will be described as being performed by a system of one or more computers located in one or more locations. For example, the patient clustering system, e.g., the patient clustering system 200 of FIG. 2, appropriately programmed in accordance with this specification, can perform the process 600d.

At 610d, the system determines, for each patient cluster, a centroid of biomedical data embeddings of patients included in the patient cluster. At 620d, the system determines, for each patient in each patient cluster, the centrality score for the patient based at least in part on a distance between: (i) the biomedical data embedding of the patient, and (ii) the centroid of the patient cluster that includes the patient.

Details of the operations of process 600d are further described with reference to FIG. 2.

FIG. 7 is a flow diagram illustrating an example process 700 for training a patient classification machine learning model. The patient classification machine learning model is configured to process an input characterizing the biomedical data of a particular patient to generate an output that characterizes a classification of the patient, e.g., which of the set of clusters (categories or classes) the particular patient belongs to.

For convenience, the process 700 will be described as being performed by a system of one or more computers located in one or more locations. For example, a classifier training system, e.g., the classifier training system 320 of FIG. 1, appropriately programmed in accordance with this specification, can perform the process 700.

At 710, the system obtains core patient data. The core patient data includes, for each of a set of patient clusters, a set of biomedical data characterizing each of a set of core patients within the patient cluster. The core patient data can be obtained, for example, from the output of a patient cluster system, such as the patient clustering system 200 with reference to FIG. 2.

At 720, the system generates a set of training examples based on the core patient data. In particular, the system generates the training examples exclusively from the data of patients identified as core patients within the population. That is, the training examples do not include data from patients who have been identified as non-core patients.

Each training example corresponds to a core patient from the population of patients, and each training example includes: (i) a training input that includes the set of biomedical data characterizing the core patient, and (ii) a target output that includes a label that identifies the patient cluster that includes the core patient.

At 730, the system trains a classification machine learning model on the set of training examples. Further details and examples of the classification machine learning model and the training of the model are described with reference to FIG. 1. In some cases, the classification machine learning model is trained subject to a constraint that classifications generated by the classification machine learning model depend on at most a predefined, maximum number of biomedical features, such as two, or three, or four, or five biomedical features. In some cases, the classification machine learning model is a decision tree, and the constraint defines a maximum depth of the decision tree.

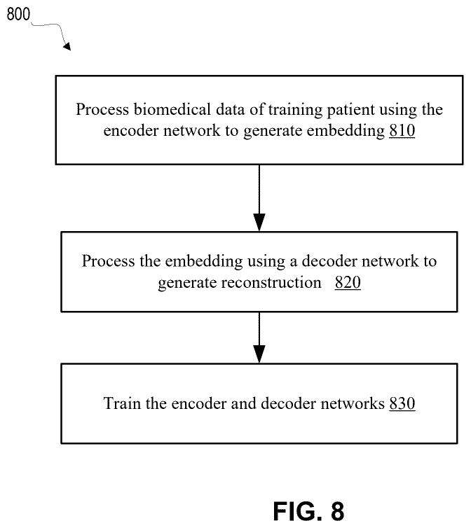

FIG. 8 is a flow diagram illustrating an example process 800 for training an encoder neural network on data from a plurality of training patients. For convenience, the process 800 will be described as being performed by a system of one or more computers located in one or more locations. For example, an encoder training system, e.g., the encoder training system 300 of FIG. 3, appropriately programmed in accordance with this specification, can perform the process 800.

At 810, the system processes, for each training patient, a set of biomedical data characterizing the training patient using the encoder neural network to generate an embedding in a latent space. At 820, the system processes the embedding for the training patient using a decoder neural network to generate a reconstruction of the set of biomedical data characterizing the training patient.

At 830, the system trains the encoder neural network and the decoder neural network to optimize an objective function that measures an error in the reconstruction of the set of biomedical data characterizing the training patient. The system can optimize the objective function using any appropriate machine learning training technique, e.g., stochastic gradient descent.

Details of the operations of training process 800 are further described with reference to FIG. 3.

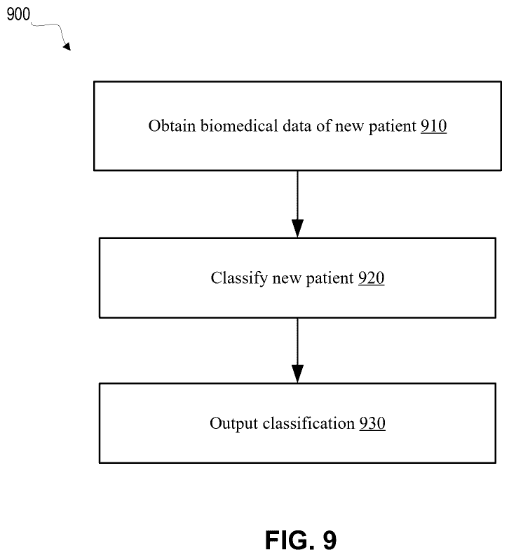

FIG. 9 is a flow diagram of an example process 900 for classifying a patient. For convenience, the process 900 will be described as being performed by a system of one or more computers located in one or more locations. For example, a classification system, e.g., the classification system 310 of FIG. 1, appropriately programmed in accordance with this specification, can perform the process 900.

At 910, the system receives a set of biomedical data characterizing a new patient. As described in further details with reference to FIG. 1, the biomedical data can include multi-modal data that includes a respective feature representation for each modality in a set of multiple modalities for the patient.

At 920, the system processes the set of biomedical data characterizing the new patient using a classification machine learning model to classify the new patient as being included in a patient cluster from the set of patient clusters. As described in further detail with reference to FIG. 1, the classification machine learning model has been trained on data from core patients identified by a patient clustering system (e.g., the system 200 with reference to FIG. 2). In some cases, the classification machine learning model can be a decision tree model with a predefined maximum depth.

At 930, the system outputs the classification generated by the classification machine learning model. As described in further detail with reference to FIG. 1, the system or another system can use the classification output in a number of different ways. For example, the system can generate a recommendation for clinical treatment of the new patient based at least in part on the classification of the new patient generated using the classification machine learning model. In some cases, a drug to the new patient can be administered to the new patient based at least in part on the classification of the new patient generated using the classification machine learning model.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using any appropriate machine learning framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
selecting, for each patient cluster in a set of patient clusters, a proper subset of a plurality of patients included in the patient cluster as core patients for the patient cluster, comprising:
generating, for each patient included in the patient cluster, a centrality score characterizing how closely the patient is associated with the patient cluster based on a biomedical data embedding associated with the patient; and
selecting the proper subset of the plurality of patients included in the patient cluster as core patients for the patient cluster based on the centrality scores; and
outputting data identifying: (i) the set of patient clusters, and (ii) the core patients for each patient cluster.

2. The method of claim 1, wherein the set of patient clusters are generated by operations comprising:
receiving, for each patient in a population of patients, a set of biomedical data characterizing the patient;
processing, for each patient in the population of patients, the set of biomedical data characterizing the patient using an encoder machine learning model to generate a biomedical data embedding of the set of biomedical data in a latent space; and
clustering the patients in the population of patients, based on the respective biomedical data embedding associated with each patient, to identify the set of patient clusters.

3. The method of claim 1, further comprising:
determining, for each patient, a measure of stability of an assignment of the patient to the patient cluster that includes the patient over a plurality of instances of clustering;
wherein for each patient in each patient cluster, the centrality score for the patient is based at least in part on the stability of the assignment of the patient to the patient cluster that includes the patient.

4. The method of claim 3, wherein determining, for each patient, the measure of stability of the assignment of the patient to the patient cluster that includes the patient over the plurality of instances of the clustering comprises:
performing the plurality of instances of the clustering, wherein each instance of the clustering generates a respective set of patient clusters; and
determining, for each patient, the measure of stability based on a measure of overlap between the patient clusters that include the patient over the plurality of instances of the clustering.

5. The method of claim 1, further comprising:
training a discriminative machine learning model to process data characterizing a patient to generate a discriminative output that classifies the patient as being included in a respective one of the patient clusters from the set of patient clusters; and
wherein for each patient in each patient cluster, generating the centrality score for the patient comprises:
determining a confidence measure that characterizes a confidence of the trained discriminative machine learning model in classifying the patient as being included in the patient cluster; and
determining the centrality score for the patient based on the confidence measure.

6. The method of claim 1, further comprising:
determining, for each patient cluster, parameters of a distribution function that characterizes a distribution of biomedical data embeddings of patients included in the patient cluster; and
determining, for each patient in each patent cluster, the centrality score for the patient based at least in part on a likelihood of the biomedical data embedding of the patient under the distribution function for the patient cluster.

7. The method of claim 1, further comprising:
determining, for each patient cluster, a centroid of biomedical data embeddings of patients included in the patient cluster; and
determining, for each patient in each patient cluster, the centrality score for the patient based at least in part on a distance between: (i) the biomedical data embedding of the patient, and (ii) the centroid of the patient cluster that includes the patient.

8. The method of claim 1, further comprising:
generating a set of training examples based on only core patients in the population of patients, wherein:
each training example corresponds to a core patient from the population of patients;
each training example comprises: (i) a training input that includes the set of biomedical data characterizing the core patient, and (ii) a target output that includes a label that identifies the patient cluster that includes the core patient; and
training a classification machine learning model on the set of training examples.

9. The method of claim 8, further comprising:
receiving a set of biomedical data characterizing a new patient;
processing the set of biomedical data characterizing the new patient using the classification machine learning model to classify the new patient as being included in a patient cluster from the set of patient clusters.

10. The method of claim 9, further comprising:
generating a recommendation for clinical treatment of the new patient based at least in part on the classification of the new patient generated using the classification machine learning model.

11. The method of claim 9, further comprising:
administering a drug to the new patient based at least in part on the classification of the new patient generated using the classification machine learning model.

12. The method of claim 8, wherein the classification machine learning model is trained subject to a constraint that classifications generated by the classification machine learning model depend on at most a predefined, maximum number of biomedical features.

13. The method of claim 12, wherein the maximum number of biomedical features is two, or three, or four, or five.

14. The method of claim 12, wherein the classification machine learning model is a decision tree, and the constraint defines a maximum depth of the decision tree.

15. The method of claim 1, further comprising:
determining, for each patient cluster, a set of statistics that characterize the patient cluster based on only the core patients of the patient cluster.

16. The method of claim 2, wherein the encoder machine learning model comprises an encoder neural network.

17. The method of claim 16, wherein the encoder neural network has been trained by operations comprising, for each of a plurality of training patients:
processing a set of biomedical data characterizing the training patient using the encoder neural network to generate an embedding in a latent space;
processing the embedding using a decoder neural network to generate a reconstruction of the set of biomedical data characterizing the training patient; and
training the encoder neural network and the decoder neural network to optimize an objective function that measures an error in the reconstruction of the set of biomedical data characterizing the training patient.

18. The method of claim 1, wherein for each patient, the set of biomedical data characterizing the patient comprises respective feature dimensions representing each of a plurality of modalities.

19. A system comprising:
one or more computers; and
one or more storage devices communicatively coupled to the one or more computers, wherein the one or more storage devices store instructions that, when executed by the one or more computers, cause the one or more computers to perform operations comprising:
selecting, for each patient cluster in a set of patient clusters, a proper subset of a plurality of patients included in the patient cluster as core patients for the patient cluster, comprising:
generating, for each patient included in the patient cluster, a centrality score characterizing how closely the patient is associated with the patient cluster based on a biomedical data embedding associated with the patient; and
selecting the proper subset of the plurality of patients included in the patient cluster as core patients for the patient cluster based on the centrality scores; and
outputting data identifying: (i) the set of patient clusters, and (ii) the core patients for each patient cluster.

20. One or more non-transitory computer storage media storing instructions that when executed by one or more computers cause the one or more computers to perform operations comprising:
selecting, for each patient cluster in a set of patient clusters, a proper subset of a plurality of patients included in the patient cluster as core patients for the patient cluster, comprising:
generating, for each patient included in the patient cluster, a centrality score characterizing how closely the patient is associated with the patient cluster based on a biomedical data embedding associated with the patient; and
selecting the proper subset of the plurality of patients included in the patient cluster as core patients for the patient cluster based on the centrality scores; and
outputting data identifying: (i) the set of patient clusters, and (ii) the core patients for each patient cluster.
